SEQUENCE LISTING (226 sequences)

SEQ ID NO: 1 - DNA, 10 bp, Artificial Sequence
  DNA oligo for barcoding
accaataata

SEQ ID NO: 2 - DNA, 10 bp, Artificial Sequence
  DNA oligo for barcoding
aataaaccta

SEQ ID NO: 3 - DNA, 10 bp, Artificial Sequence
  DNA oligo for barcoding
acatcatcat

SEQ ID NO: 4-53 - skipped (50 entries)

SEQ ID NO: 54 - DNA, 20 bp, Artificial Sequence
  DNA oligo for barcoding
gagtgcggcctttgattaaa

SEQ ID NO: 55 - DNA, 20 bp, Artificial Sequence
  DNA oligo for barcoding
ctccgttacaaccgtcacgt

SEQ ID NO: 56 - DNA, 20 bp, Artificial Sequence
  DNA oligo for barcoding
tttggtgcaatcaccgccca

SEQ ID NO: 57 - DNA, 20 bp, Artificial Sequence
  DNA oligo for barcoding
cgtgtaatccgggagcaaca

SEQ ID NO: 58 - DNA, 20 bp, Artificial Sequence
  DNA oligo for barcoding
tcctatgtcgcgtcattgcc

SEQ ID NO: 59 - DNA, 20 bp, Artificial Sequence
  DNA oligo for barcoding
tctttgtgcgtggccctcca

SEQ ID NO: 60 - DNA, 20 bp, Artificial Sequence
  DNA oligo for barcoding
acatcctgaacaccgcggga

SEQ ID NO: 61 - DNA, 20 bp, Artificial Sequence
  DNA oligo for barcoding
attggcgagaattcatgctc

SEQ ID NO: 62 - DNA, 20 bp, Artificial Sequence
  DNA oligo for barcoding
gtccgacatacctctaacga

SEQ ID NO: 63 - DNA, 20 bp, Artificial Sequence
  DNA oligo for barcoding
aagtaggttcaccctgccgg

SEQ ID NO: 64 - DNA, 20 bp, Artificial Sequence
  DNA oligo for barcoding
aagcgcgaagatgcaattgc

SEQ ID NO: 65 - DNA, 20 bp, Artificial Sequence
  DNA oligo for barcoding
aatcacaggccgagctcaga

SEQ ID NO: 66 - DNA, 20 bp, Artificial Sequence
  DNA oligo for barcoding
ttacgaaattccaccctcgc

SEQ ID NO: 67 - DNA, 20 bp, Artificial Sequence
  DNA oligo for barcoding
tgtttcactcagtaagccct

SEQ ID NO: 68 - DNA, 20 bp, Artificial Sequence
  DNA oligo for barcoding
cattattgcgctggcccgta

SEQ ID NO: 69 - DNA, 20 bp, Artificial Sequence
  DNA oligo for barcoding
ggttagcgttatcaccagtg

SEQ ID NO: 70 - DNA, 20 bp, Artificial Sequence
  DNA oligo for barcoding
gaacagattacttgcggtcg

SEQ ID NO: 71 - DNA, 20 bp, Artificial Sequence
  DNA oligo for barcoding
gtgaggtcggttgcgatata

SEQ ID NO: 72 - DNA, 20 bp, Artificial Sequence
  DNA oligo for barcoding
gacgacctgcggatgaatgg

SEQ ID NO: 73 - DNA, 20 bp, Artificial Sequence
  DNA oligo for barcoding
agcataccggccttgagtcc

SEQ ID NO: 74 - DNA, 20 bp, Artificial Sequence
  DNA oligo for barcoding
cgtacgctgtataaggccag

SEQ ID NO: 75 - DNA, 20 bp, Artificial Sequence
  DNA oligo for barcoding
agtacgcccataacggatag

SEQ ID NO: 76 - DNA, 20 bp, Artificial Sequence
  DNA oligo for barcoding
cttgatacgccgggaccaca

SEQ ID NO: 77 - DNA, 20 bp, Artificial Sequence
  DNA oligo for barcoding
tcaacgctgtacgagtgctg

SEQ ID NO: 78 - DNA, 20 bp, Artificial Sequence
  DNA oligo for barcoding
gatgcatccaatcagtcatc

SEQ ID NO: 79 - DNA, 20 bp, Artificial Sequence
  DNA oligo for barcoding
tgacggactgctagcccaac

SEQ ID NO: 80 - DNA, 20 bp, Artificial Sequence
  DNA oligo for barcoding
aacatagggtttattgcgcc

SEQ ID NO: 81 - DNA, 20 bp, Artificial Sequence
  DNA oligo for barcoding
cgaactacctctaggacgac

SEQ ID NO: 82 - DNA, 20 bp, Artificial Sequence
  DNA oligo for barcoding
acataggcgtacgagggaat

SEQ ID NO: 83 - DNA, 20 bp, Artificial Sequence
  DNA oligo for barcoding
gttgcatcccgtctggattc

SEQ ID NO: 84 - DNA, 20 bp, Artificial Sequence
  DNA oligo for barcoding
ataatgtgaatctcgtgcgg

SEQ ID NO: 85 - DNA, 20 bp, Artificial Sequence
  DNA oligo for barcoding
gttggatccaggcggaacag

SEQ ID NO: 86 - DNA, 20 bp, Artificial Sequence
  DNA oligo for barcoding
tagaactgacgtccgcttat

SEQ ID NO: 87 - DNA, 20 bp, Artificial Sequence
  DNA oligo for barcoding
cagattagagcttccgtatc

SEQ ID NO: 88 - DNA, 20 bp, Artificial Sequence
  DNA oligo for barcoding
tatggccgcgaattctagac

SEQ ID NO: 89 - DNA, 20 bp, Artificial Sequence
  DNA oligo for barcoding
tcacagttcgcgtatcagag

SEQ ID NO: 90 - DNA, 20 bp, Artificial Sequence
  DNA oligo for barcoding
aagcgccgtatcctgattga

SEQ ID NO: 91 - DNA, 20 bp, Artificial Sequence
  DNA oligo for barcoding
tgtgcatatccgagacctga

SEQ ID NO: 92 - DNA, 20 bp, Artificial Sequence
  DNA oligo for barcoding
gacattgtcgtacgcagcta

SEQ ID NO: 93 - DNA, 20 bp, Artificial Sequence
  DNA oligo for barcoding
aatcaggcgcacgttgtcat

SEQ ID NO: 94 - DNA, 20 bp, Artificial Sequence
  DNA oligo for barcoding
atcacggcgacatcttaggt

SEQ ID NO: 95 - DNA, 20 bp, Artificial Sequence
  DNA oligo for barcoding
cgccacgtcgtatgataagt

SEQ ID NO: 96 - DNA, 20 bp, Artificial Sequence
  DNA oligo for barcoding
tcggccgtagaatgatccat

SEQ ID NO: 97 - DNA, 20 bp, Artificial Sequence
  DNA oligo for barcoding
tccttgcgtgagactagaca

SEQ ID NO: 98 - DNA, 20 bp, Artificial Sequence
  DNA oligo for barcoding
gaatgtcgcgccaatctatg

SEQ ID NO: 99 - DNA, 20 bp, Artificial Sequence
  DNA oligo for barcoding
acctggcgcaatagttactg

SEQ ID NO: 100 - DNA, 20 bp, Artificial Sequence
  DNA oligo for barcoding
taggtcaggcttcgtcaaca

SEQ ID NO: 101 - DNA, 20 bp, Artificial Sequence
  DNA oligo for barcoding
gatatccgcgcgtagttaac

SEQ ID NO: 102 - DNA, 20 bp, Artificial Sequence
  DNA oligo for barcoding
gtaatcgctcgcccatgaca

SEQ ID NO: 103 - DNA, 20 bp, Artificial Sequence
  DNA oligo for barcoding
gtactggacctcgcacaatg

SEQ ID NO: 104 - DNA, 20 bp, Artificial Sequence
  DNA oligo for barcoding
actgagcgcccaattgatct

SEQ ID NO: 105 - DNA, 20 bp, Artificial Sequence
  DNA oligo for barcoding
tacgtacgatcctagagccc

SEQ ID NO: 106 - DNA, 20 bp, Artificial Sequence
  DNA oligo for barcoding
tttcccagtgcgagtcaaga

SEQ ID NO: 107 - DNA, 20 bp, Artificial Sequence
  DNA oligo for barcoding
gcccagcctccgtaataatg

SEQ ID NO: 108 - DNA, 20 bp, Artificial Sequence
  DNA oligo for barcoding
gcaaccctgtcgagctaagt

SEQ ID NO: 109 - DNA, 20 bp, Artificial Sequence
  DNA oligo for barcoding
tctataagaggtccgcatcg

SEQ ID NO: 110 - DNA, 20 bp, Artificial Sequence
  DNA oligo for barcoding
aatccaggtgcctagcgatt

SEQ ID NO: 111 - DNA, 20 bp, Artificial Sequence
  DNA oligo for barcoding
ctcttagtcaatgccgcaca

SEQ ID NO: 112 - DNA, 20 bp, Artificial Sequence
  DNA oligo for barcoding
tatctatagccgcccgagac

SEQ ID NO: 113 - DNA, 20 bp, Artificial Sequence
  DNA oligo for barcoding
tgcaatcctggcgaacactc

SEQ ID NO: 114 - DNA, 21 bp, Artificial Sequence
  DNA oligo for barcoding
tttattattggtattattggt

SEQ ID NO: 115 - DNA, 21 bp, Artificial Sequence
  DNA oligo for barcoding
tttaggtttattaggtttatt

SEQ ID NO: 116 - DNA, 21 bp, Artificial Sequence
  DNA oligo for barcoding
ttatgatgatgatgatgatgt

SEQ ID NO: 117 - DNA, 21 bp, Artificial Sequence
  DNA oligo for barcoding
ttgttaagttggttaagttgt

SEQ ID NO: 118 - DNA, 21 bp, Artificial Sequence
  DNA oligo for barcoding
ttgattttagagattttagat

SEQ ID NO: 119 - DNA, 21 bp, Artificial Sequence
  DNA oligo for barcoding
ttgagagtattgagagtattt

SEQ ID NO: 120 - DNA, 21 bp, Artificial Sequence
  DNA oligo for barcoding
ttagtgaataaagtgaataat

SEQ ID NO: 121 - DNA, 21 bp, Artificial Sequence
  DNA oligo for barcoding
ttgaaaaaaaggaaaaaaagt

SEQ ID NO: 122 - DNA, 21 bp, Artificial Sequence
  DNA oligo for barcoding
ttaatagaaggaatagaaggt

SEQ ID NO: 123 - DNA, 21 bp, Artificial Sequence
  DNA oligo for barcoding
ttgtagtagaggtagtagagt

SEQ ID NO: 124 - DNA, 21 bp, Artificial Sequence
  DNA oligo for barcoding
ttgagtttttagagtttttat

SEQ ID NO: 125 - DNA, 21 bp, Artificial Sequence
  DNA oligo for barcoding
ttagattagttagattagttt

SEQ ID NO: 126 - DNA, 21 bp, Artificial Sequence
  DNA oligo for barcoding
ttgaagagaaagaagagaaat

SEQ ID NO: 127 - DNA, 21 bp, Artificial Sequence
  DNA oligo for barcoding
tttagtatgtttagtatgttt

SEQ ID NO: 128 - DNA, 21 bp, Artificial Sequence
  DNA oligo for barcoding
ttgtaaatgaagtaaatgaat

SEQ ID NO: 129 - DNA, 21 bp, Artificial Sequence
  DNA oligo for barcoding
ttttgtaggatttgtaggatt

SEQ ID NO: 130 - DNA, 21 bp, Artificial Sequence
  DNA oligo for barcoding
ttttttgattgttttgattgt

SEQ ID NO: 131 - DNA, 21 bp, Artificial Sequence
  DNA oligo for barcoding
ttgtttgtaaggtttgtaagt

SEQ ID NO: 132 - DNA, 21 bp, Artificial Sequence
  DNA oligo for barcoding
ttgttatttgtgttatttgtt

SEQ ID NO: 133 - DNA, 21 bp, Artificial Sequence
  DNA oligo for barcoding
ttggtagaaaaggtagaaaat

SEQ ID NO: 134 - DNA, 21 bp, Artificial Sequence
  DNA oligo for barcoding
ttaaataagggaaataagggt

SEQ ID NO: 135 - DNA, 21 bp, Artificial Sequence
  DNA oligo for barcoding
ttaatgaaagaaatgaaagat

SEQ ID NO: 136 - DNA, 21 bp, Artificial Sequence
  DNA oligo for barcoding
ttgagtaagaagagtaagaat

SEQ ID NO: 137 - DNA, 21 bp, Artificial Sequence
  DNA oligo for barcoding
ttgatttatgggatttatggt

SEQ ID NO: 138 - DNA, 21 bp, Artificial Sequence
  DNA oligo for barcoding
ttggataaatgggataaatgt

SEQ ID NO: 139 - DNA, 21 bp, Artificial Sequence
  DNA oligo for barcoding
ttgtgaagtatgtgaagtatt

SEQ ID NO: 140 - DNA, 21 bp, Artificial Sequence
  DNA oligo for barcoding
ttttagaggtattagaggtat

SEQ ID NO: 141 - DNA, 21 bp, Artificial Sequence
  DNA oligo for barcoding
ttaaataggagaaataggagt

SEQ ID NO: 142 - DNA, 21 bp, Artificial Sequence
  DNA oligo for barcoding
tttttggatagtttggatagt

SEQ ID NO: 143 - DNA, 21 bp, Artificial Sequence
  DNA oligo for barcoding
ttgatagggatgatagggatt

SEQ ID NO: 144 - DNA, 21 bp, Artificial Sequence
  DNA oligo for barcoding
ttaagtaatgaaagtaatgat

SEQ ID NO: 145 - DNA, 21 bp, Artificial Sequence
  DNA oligo for barcoding
ttgagatttaggagatttagt

SEQ ID NO: 146 - DNA, 21 bp, Artificial Sequence
  DNA oligo for barcoding
ttaaaagtagtaaaagtagtt

SEQ ID NO: 147 - DNA, 21 bp, Artificial Sequence
  DNA oligo for barcoding
ttgagatagtagagatagtat

SEQ ID NO: 148 - DNA, 21 bp, Artificial Sequence
  DNA oligo for barcoding
ttggaagatatggaagatatt

SEQ ID NO: 149 - DNA, 21 bp, Artificial Sequence
  DNA oligo for barcoding
ttatagttagtatagttagtt

SEQ ID NO: 150 - DNA, 21 bp, Artificial Sequence
  DNA oligo for barcoding
ttagttgataaagttgataat

SEQ ID NO: 151 - DNA, 21 bp, Artificial Sequence
  DNA oligo for barcoding
ttgaaaagttagaaaagttat

SEQ ID NO: 152 - DNA, 21 bp, Artificial Sequence
  DNA oligo for barcoding
ttatgtaaagaatgtaaagat

SEQ ID NO: 153 - DNA, 21 bp, Artificial Sequence
  DNA oligo for barcoding
ttaagtataggaagtataggt

SEQ ID NO: 154 - DNA, 21 bp, Artificial Sequence
  DNA oligo for barcoding
ttaaagaagaaaaagaagaat

SEQ ID NO: 155 - DNA, 21 bp, Artificial Sequence
  DNA oligo for barcoding
ttattatgtgaattatgtgat

SEQ ID NO: 156 - DNA, 21 bp, Artificial Sequence
  DNA oligo for barcoding
tttgatatgattgatatgatt

SEQ ID NO: 157 - DNA, 21 bp, Artificial Sequence
  DNA oligo for barcoding
ttagatagaaaagatagaaat

SEQ ID NO: 158 - DNA, 21 bp, Artificial Sequence
  DNA oligo for barcoding
ttataaaaggaataaaaggat

SEQ ID NO: 159 - DNA, 21 bp, Artificial Sequence
  DNA oligo for barcoding
ttggtataagaggtataagat

SEQ ID NO: 160 - DNA, 21 bp, Artificial Sequence
  DNA oligo for barcoding
tttgtaatatgtgtaatatgt

SEQ ID NO: 161 - DNA, 21 bp, Artificial Sequence
  DNA oligo for barcoding
ttgattaggaagattaggaat

SEQ ID NO: 162 - DNA, 21 bp, Artificial Sequence
  DNA oligo for barcoding
tttgtagattatgtagattat

SEQ ID NO: 163 - DNA, 21 bp, Artificial Sequence
  DNA oligo for barcoding
ttagatttttaagatttttat

SEQ ID NO: 164 - DNA, 23 bp, Artificial Sequence
  DNA oligo for barcoding
tttttaatcaaaggccgcactct

SEQ ID NO: 165 - DNA, 23 bp, Artificial Sequence
  DNA oligo for barcoding
ttacgtgacggttgtaacggagt

SEQ ID NO: 166 - DNA, 23 bp, Artificial Sequence
  DNA oligo for barcoding
tttgggcggtgattgcaccaaat

SEQ ID NO: 167 - DNA, 23 bp, Artificial Sequence
  DNA oligo for barcoding
tttgttgctcccggattacacgt

SEQ ID NO: 168 - DNA, 23 bp, Artificial Sequence
  DNA oligo for barcoding
ttggcaatgacgcgacataggat

SEQ ID NO: 169 - DNA, 23 bp, Artificial Sequence
  DNA oligo for barcoding
tttggagggccacgcacaaagat

SEQ ID NO: 170 - DNA, 23 bp, Artificial Sequence
  DNA oligo for barcoding
tttcccgcggtgttcaggatgtt

SEQ ID NO: 171 - DNA, 23 bp, Artificial Sequence
  DNA oligo for barcoding
ttgagcatgaattctcgccaatt

SEQ ID NO: 172 - DNA, 23 bp, Artificial Sequence
  DNA oligo for barcoding
tttcgttagaggtatgtcggact

SEQ ID NO: 173 - DNA, 23 bp, Artificial Sequence
  DNA oligo for barcoding
ttccggcagggtgaacctacttt

SEQ ID NO: 174 - DNA, 23 bp, Artificial Sequence
  DNA oligo for barcoding
ttgcaattgcatcttcgcgcttt

SEQ ID NO: 175 - DNA, 23 bp, Artificial Sequence
  DNA oligo for barcoding
tttctgagctcggcctgtgattt

SEQ ID NO: 176 - DNA, 23 bp, Artificial Sequence
  DNA oligo for barcoding
ttgcgagggtggaatttcgtaat

SEQ ID NO: 177 - DNA, 23 bp, Artificial Sequence
  DNA oligo for barcoding
ttagggcttactgagtgaaacat

SEQ ID NO: 178 - DNA, 23 bp, Artificial Sequence
  DNA oligo for barcoding
tttacgggccagcgcaataatgt

SEQ ID NO: 179 - DNA, 23 bp, Artificial Sequence
  DNA oligo for barcoding
ttcactggtgataacgctaacct

SEQ ID NO: 180 - DNA, 23 bp, Artificial Sequence
  DNA oligo for barcoding
ttcgaccgcaagtaatctgttct

SEQ ID NO: 181 - DNA, 23 bp, Artificial Sequence
  DNA oligo for barcoding
tttatatcgcaaccgacctcact

SEQ ID NO: 182 - DNA, 23 bp, Artificial Sequence
  DNA oligo for barcoding
ttccattcatccgcaggtcgtct

SEQ ID NO: 183 - DNA, 23 bp, Artificial Sequence
  DNA oligo for barcoding
ttggactcaaggccggtatgctt

SEQ ID NO: 184 - DNA, 23 bp, Artificial Sequence
  DNA oligo for barcoding
ttctggccttatacagcgtacgt

SEQ ID NO: 185 - DNA, 23 bp, Artificial Sequence
  DNA oligo for barcoding
ttctatccgttatgggcgtactt

SEQ ID NO: 186 - DNA, 23 bp, Artificial Sequence
  DNA oligo for barcoding
tttgtggtcccggcgtatcaagt

SEQ ID NO: 187 - DNA, 23 bp, Artificial Sequence
  DNA oligo for barcoding
ttcagcactcgtacagcgttgat

SEQ ID NO: 188 - DNA, 23 bp, Artificial Sequence
  DNA oligo for barcoding
ttgatgactgattggatgcatct

SEQ ID NO: 189 - DNA, 23 bp, Artificial Sequence
  DNA oligo for barcoding
ttgttgggctagcagtccgtcat

SEQ ID NO: 190 - DNA, 23 bp, Artificial Sequence
  DNA oligo for barcoding
ttggcgcaataaaccctatgttt

SEQ ID NO: 191 - DNA, 23 bp, Artificial Sequence
  DNA oligo for barcoding
ttgtcgtcctagaggtagttcgt

SEQ ID NO: 192 - DNA, 23 bp, Artificial Sequence
  DNA oligo for barcoding
ttattccctcgtacgcctatgtt

SEQ ID NO: 193 - DNA, 23 bp, Artificial Sequence
  DNA oligo for barcoding
ttgaatccagacgggatgcaact

SEQ ID NO: 194 - DNA, 23 bp, Artificial Sequence
  DNA oligo for barcoding
ttccgcacgagattcacattatt

SEQ ID NO: 195 - DNA, 23 bp, Artificial Sequence
  DNA oligo for barcoding
ttctgttccgcctggatccaact

SEQ ID NO: 196 - DNA, 23 bp, Artificial Sequence
  DNA oligo for barcoding
ttataagcggacgtcagttctat

SEQ ID NO: 197 - DNA, 23 bp, Artificial Sequence
  DNA oligo for barcoding
ttgatacggaagctctaatctgt

SEQ ID NO: 198 - DNA, 23 bp, Artificial Sequence
  DNA oligo for barcoding
ttgtctagaattcgcggccatat

SEQ ID NO: 199 - DNA, 23 bp, Artificial Sequence
  DNA oligo for barcoding
ttctctgatacgcgaactgtgat

SEQ ID NO: 200 - DNA, 23 bp, Artificial Sequence
  DNA oligo for barcoding
tttcaatcaggatacggcgcttt

SEQ ID NO: 201 - DNA, 23 bp, Artificial Sequence
  DNA oligo for barcoding
tttcaggtctcggatatgcacat

SEQ ID NO: 202 - DNA, 23 bp, Artificial Sequence
  DNA oligo for barcoding
tttagctgcgtacgacaatgtct

SEQ ID NO: 203 - DNA, 23 bp, Artificial Sequence
  DNA oligo for barcoding
ttatgacaacgtgcgcctgattt

SEQ ID NO: 204 - DNA, 23 bp, Artificial Sequence
  DNA oligo for barcoding
ttacctaagatgtcgccgtgatt

SEQ ID NO: 205 - DNA, 23 bp, Artificial Sequence
  DNA oligo for barcoding
ttacttatcatacgacgtggcgt

SEQ ID NO: 206 - DNA, 23 bp, Artificial Sequence
  DNA oligo for barcoding
ttatggatcattctacggccgat

SEQ ID NO: 207 - DNA, 23 bp, Artificial Sequence
  DNA oligo for barcoding
tttgtctagtctcacgcaaggat

SEQ ID NO: 208 - DNA, 23 bp, Artificial Sequence
  DNA oligo for barcoding
ttcatagattggcgcgacattct

SEQ ID NO: 209 - DNA, 23 bp, Artificial Sequence
  DNA oligo for barcoding
ttcagtaactattgcgccaggtt

SEQ ID NO: 210 - DNA, 23 bp, Artificial Sequence
  DNA oligo for barcoding
tttgttgacgaagcctgacctat

SEQ ID NO: 211 - DNA, 23 bp, Artificial Sequence
  DNA oligo for barcoding
ttgttaactacgcgcggatatct

SEQ ID NO: 212 - DNA, 23 bp, Artificial Sequence
  DNA oligo for barcoding
tttgtcatgggcgagcgattact

SEQ ID NO: 213 - DNA, 23 bp, Artificial Sequence
  DNA oligo for barcoding
ttcattgtgcgaggtccagtact

SEQ ID NO: 214 - DNA, 23 bp, Artificial Sequence
  DNA oligo for barcoding
ttagatcaattgggcgctcagtt

SEQ ID NO: 215 - DNA, 23 bp, Artificial Sequence
  DNA oligo for barcoding
ttgggctctaggatcgtacgtat

SEQ ID NO: 216 - DNA, 23 bp, Artificial Sequence
  DNA oligo for barcoding
tttcttgactcgcactgggaaat

SEQ ID NO: 217 - DNA, 23 bp, Artificial Sequence
  DNA oligo for barcoding
ttcattattacggaggctgggct

SEQ ID NO: 218 - DNA, 23 bp, Artificial Sequence
  DNA oligo for barcoding
ttacttagctcgacagggttgct

SEQ ID NO: 219 - DNA, 23 bp, Artificial Sequence
  DNA oligo for barcoding
ttcgatgcggacctcttatagat

SEQ ID NO: 220 - DNA, 23 bp, Artificial Sequence
  DNA oligo for barcoding
ttaatcgctaggcacctggattt

SEQ ID NO: 221 - DNA, 23 bp, Artificial Sequence
  DNA oligo for barcoding
tttgtgcggcattgactaagagt

SEQ ID NO: 222 - DNA, 23 bp, Artificial Sequence
  DNA oligo for barcoding
ttgtctcgggcggctatagatat

SEQ ID NO: 223 - DNA, 23 bp, Artificial Sequence
  DNA oligo for barcoding
ttgagtgttcgccaggattgcat

SEQ ID NO: 224 - DNA, 22 bp, Artificial Sequence
  complementary fluorescent-labeled DNA oligo
  misc_feature (1)..(1): 5′ end modification: FAM
tttattattggttattattggt

SEQ ID NO: 225 - DNA, 22 bp, Artificial Sequence
  complementary fluorescent-labeled DNA oligo
  misc_feature (1)..(1): 5′ end modification: TexasRed
tttaggtttatttaggtttatt

SEQ ID NO: 226 - DNA, 22 bp, Artificial Sequence
  complementary fluorescent-labeled DNA oligo
  misc_feature (1)..(1): 5′ end modification: Cy5.5
ttatgatgatgtatgatgatgt

(19) United States
(12) Patent Application Publication
(10) Pub. No.: US 2023/0194509 A1
(43) Pub. Date: Jun. 22, 2023

(54) EFFICIENT ANTIBODY DNA-BARCODING REAGENTS FOR MULTIPLEXED MOLECULAR IMAGING

(71) Applicant: The Chinese University of Hong Kong, Hong Kong (CN)

(72) Inventors: Ho KO, Hong Kong (CN); Hei Ming LAI, Hong Kong (CN)

(21) Appl. No.: 17/999,154

(22) PCT Filed: Jun. 18, 2021
(86) PCT No.: PCT/CN2021/100919

§ 371 Date: Nov. 17, 2022

Related U.S. Application Data
(60) Provisional application No. 63/040,557, filed on Jun. 18, 2020.

(51) Int. Cl. G01N 33/533; C07K 16/28

(52) U.S. Cl. CPC G01N 33/533; C07K 16/2803; C07K 2317/55

(57) ABSTRACT

Provided are DNA-barcoding reagents, DNA-barcoded antibodies, and methods of using DNA-barcoding reagents, and DNA-barcoded antibodies. The DNA-barcoding reagents comprise an affinity moiety that is specific towards antibodies, and covalently linked to one or more DNA sequences, optionally, by a linker. Also provided are methods of using the DNA-barcoded antibodies, for example, multiplexed tissue antigen imaging and profiling, multiplexed biomolecule detection, and affinity purification and sorting of marker-positive cells.

### CROSS-REFERENCE TO RELATED APPLICATION

This application claims the benefit of U.S. Provisional Application Ser. No. 63/040,557, filed Jun. 18, 2020, which is hereby incorporated by reference in its entirety including any tables, figures, or drawings.

### SEQUENCE LISTING

The Sequence Listing for this application is labeled “SeqList-17June20_ST25.txt,” which was created on 17 Jun. 2020, and is 37 KB. The Sequence Listing is incorporated herein by reference in its entirety.

### BACKGROUND OF THE INVENTION

In biotechnology, simultaneously detecting, quantifying, and characterizing a large number of molecules in a single sample is highly desirable. Such multiplexed detection of biomolecules is a new trend in tissue research and diagnostics. In recent years, it has been increasingly recognized that cells and biomolecules must be understood in their environmental context and in relation to each other. Single-cell technologies have uncovered a rich heterogeneity among cell populations, which has yet to be put into spatial context in their natural tissue organization. There is thus a demand for methods that can simultaneously image the distributions of many biomolecules in tissues, or detect many biomolecules and characterizing them all at once.

Multiplexed molecular methods have been developed based on the use of primary antibodies that have been barcoded with a DNA sequence, followed by readout using complementary fluorophore-labeled DNAs (called DNA imagers) or polymerase chain reaction. DNA-barcoded antibodies are important tools for specific target binding via the antibody as well as the access to an array of DNA technologies, via the linked DNA handle, for highly multiplexed readout, programming, and amplification. For example, DNA-barcoded antibodies are well suited for use in single-cell technologies, multiplexed enzyme-linked immunosorbent assays (ELISA), multiplexed immunofluorescence for histology, super-resolution imaging, and expansion microscopy etc.

With the use of DNA-barcoded antibodies, multiplexed tissue antigen imaging can help clarify interrelationships between different molecules and cells in terms of their distributions. In multiplexed immunoassays, these methods will allow the quantification of many proteins at once. In single-cell technologies, multiplexed sorting of individual cells with certain markers can be aided by flow cytometry and the use of DNA-barcoded antibodies.

Surprisingly, there has been little progress in the synthesis of DNA-barcoded antibodies. This is very important as the above-stated applications rely on highly customized configuration, where the users must conjugate a DNA to their primary antibodies of interest, the synthetic procedure for which is the bottleneck to the widespread use of DNA-barcoded antibodies. Existing methods of conjugating DNA molecules to commercially available primary antibodies are tedious, requiring long reaction times (>16 hours), and multiple protein concentration and purification steps.

Existing approaches to DNA-barcoding a primary antibody typically involve: (1) purifying and concentrating the commercial primary antibody to remove any interfering chemicals, (2) activating the primary antibody and DNA with reactive groups, (3) purifying the activated primary antibody and DNA, and (4) reacting the activated primary antibody and DNA in a conjugation reaction, typically requiring at least 4 hours. Since the conjugation reaction is usually incomplete and has low yields, purification will require (5) removing unreacted DNAs, (6) removing unreacted primary antibodies, and (7) concentrating the product.

Steps (5)-(6) can be simultaneously achieved using high performance liquid chromatography (HPLC), but this requires expensive equipment and setup. Alternatively, step (5) can first be performed with ultrafiltration to remove the DNAs (which have lower molecular weights), then step (6) can proceed with affinity-based purification using complementary DNA-conjugated beads/column, followed by elution with specific displacing DNAs, and ultrafiltration to remove the excess displacing DNAs.

These methods are not readily accessible to most biological laboratories, are cost-inefficient, and are not user friendly. They are also inflexible because if one desires to conjugate DNA of an alternative sequence to the primary antibody, the whole synthesis procedure must be repeated. Finally, the yield of step (4) is typically low (<50%) even with optimized reaction conditions. Therefore, there is a need to design and develop novel DNA-barcoding reagent that circumvent all the above problems.

### BRIEF SUMMARY OF THE INVENTION

The subject invention provides methods for designing/synthesizing a novel DNA-barcoding reagent that do not require long reaction times and multiple protein concentration and purification steps. Advantageously, the methods for producing the DNA-barcoding reagent according to the subject are cost-efficient and user friendly.

The subject invention also provides DNA-barcoding reagents that are produced by the method of the present invention. The DNA-barcoding reagent comprises an affinity moiety that is specific towards antibodies, and covalently linked to one or more DNA sequences, optionally, by a linker.

Advantageously, the attachment mediated by the affinity moiety is robust. It is not interfered with or competed by any components present in the antibody storage buffers. The attachment process is quantitative and rapid (<10 minutes at room temperature). In practice, a slight molar excess of the DNA-barcoding reagent can be used to ensure complete labeling. After the attachment process, the excess DNA-barcoding reagent can be scavenged using non-specific immunoglobulins from the same target host species.

The subject invention further provides methods of using the DNA-barcoding reagent to label antibodies for producing DNA-barcoding antibodies, and methods of using the DNA-barcoding antibodies in, for example, single-cell technologies, ELISA, multiplexed immunofluorescence for histology, super-resolution imaging, and expansion microscopy.

In one embodiment, the subject invention provides a method for multiplexed molecular imaging in a sample, comprising contacting one or more DNA-barcoded antibodies with the sample, wherein the DNA-barcoded antibody comprising an affinity moiety specific to an antibody, wherein the antibody is covalently linked to one or more DNA sequences or via a linker, and one or more DNA sequences are labelled with one or more reporters, such as fluorophores.

With the use of DNA-barcoded antibodies, multiplexed tissue antigen imaging can help clarify interrelationships between different molecules and cells in terms of their distributions. In multiplexed immunoassays, these methods will allow the quantification of many proteins at once. In single-cell technologies, multiplexed sorting of individual cells with certain markers can be aided by flow cytometry and the use of DNA-barcoded antibodies.

The DNA-barcoding reagent according to the subject invention can be used for commercially available primary antibodies. In specific embodiments, the DNA-barcoding reagent comprises, consists essentially of, or consists of a secondary antibody Fab fragment or VHH fragment conjugated to a DNA barcode. With this reagent, commercially available antibodies can be DNA-barcoded in a single 10-minute incubation step at room temperature, achieving quantitative yields. The so-formed complex can be used directly or after simple clean up with an additional 10-minute incubation step, for many biotechnological applications without further purification.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the diagrammatic representation of the DNA-barcoding reagent.

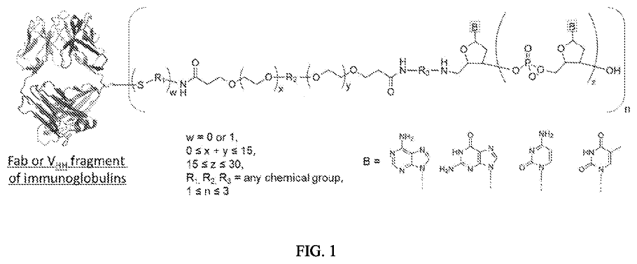

FIG. 2 shows the comparison of the existing methods and the subject invention in preparing the DNA-barcoded antibodies.

FIG. 3 shows the SDS-PAGE gel demonstrating that DNA is conjugated to Fab fragments of secondary antibodies.

FIG. 4 shows the multiplexed tissue antigen imaging in a human oral squamous cell carcinoma sample. All primary antibodies used are commercially available and raised from the same host species (Mouse, Ms), separately DNA-barcoded using the synthesized DNA-barcoding reagents, pooled without purification, and added to the tissue all at once. Imaging of the DNAs was performed using complementary fluorescent-labeled DNA oligos.

### BRIEF DESCRIPTION OF SEQUENCES

SEQ ID NOs: 1-223 are DNA sequences of the stretch of DNA molecules contemplated for use according to the subject invention.

SEQ ID NOs: 224-226 are sequences complementary fluorescent-labeled DNA oligos contemplated for use according to the subject invention.

### DETAILED DISCLOSURE OF THE INVENTION

The subject invention provides methods for designing/synthesizing a novel DNA-barcoding reagent that do not require long reaction times and multiple protein concentration and purification steps. Advantageously, the methods for producing the DNA-barcoding reagent according to the subject are cost-efficient and user friendly.

The subject invention also provides DNA-barcoding reagents that are produced by the method of the present invention. The DNA-barcoding reagent comprises an affinity moiety that is specific towards antibodies, and covalently linked to one or more DNA sequences, optionally, by a linker.

Advantageously, the attachment mediated by the affinity moiety is robust. It is not interfered with or competed by any components present in the antibody storage buffers. The attachment process is quantitative and rapid (<10 minutes at room temperature). In practice, a slight molar excess of the DNA-barcoding reagent can be used to ensure complete labeling. After the attachment process, the excess DNA-barcoding reagent can be scavenged using non-specific immunoglobulins from the same target host species.

In one embodiment, the DNA-barcoding reagent comprises an affinity moiety covalently linked to one or more DNA molecules via a flexible, water-soluble linker.

In one embodiment, the DNA-barcoding reagent comprises an affinity moiety, a DNA part, and an intervening linker part, to non-covalently attach a specific DNA sequence to an antibody, for example, selected from commercially available primary antibodies.

In one embodiment, the affinity moiety can attach one or more DNA molecules, for example, 1 to 10, 1 to 9, 1 to 8, 1 to 7, 1 to 6, 1 to 5, 1 to 4, 1 to 3, or 1 to 2 DNA molecules. Preferably, there can be 1-3 DNA molecules attached per affinity moiety.

In one embodiment, the affinity moiety can be selected from 1) a Fab fragment of an antibody originated from, for example, goat or donkey, the target of which is specific towards the immunoglobulin G (IgG) class molecules from, for example, mouse, rat, rabbit, goat, or guinea pig; and immunoglobulin Y (IgY) molecules from, for example, chicken; and 2) a modified V_{HH} domain of an antibody originated from, for example, Camelidae, the target of which is specific towards the immunoglobulin G (IgG) class molecules from, for example, mouse and rabbit. This protein molecule may be produced from a recombinant source.

In one embodiment, the subject invention also provides a DNA barcode-linker comprising a DNA part, and an intervening linker part. In a specific embodiment, the DNA barcode-linker has a structure of

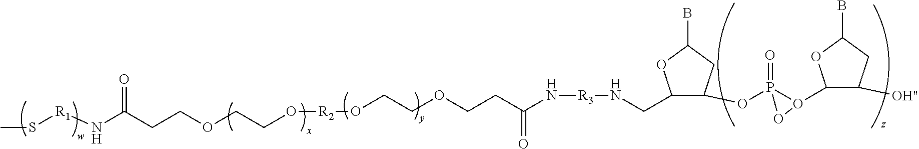

wherein W is 0 or 1; 0≤x+y≤15; 15≤z≤60; 1≤n≤10; R₁, R₂ and R₃ can be any chemical group; and B is selected from adenine

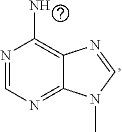

guanine

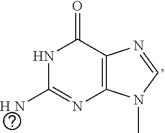

cytosine

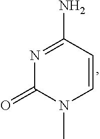

and thymine

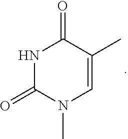

In specific embodiments, R₁, R₂ and R₃ are independently selected from, for example, O, S, alkylene, substituted alkylene, arylene, substituted arylene, heteroalkylene, substituted heteroalkylene, heteroarylene, substituted heteroarylene, cycloalkylene, substituted cycloalkylene, heterocycloalkylene, substituted heterocycloalkylene, cycloalkenylene, substituted cycloalkenylene, alkenylene, substituted alkenylene, alkynylene, substituted alkynylene, —NR₄—, —CO—, and —COO—, wherein R₄ is selected from, for example, hydrogen, alkyl, substituted alkyl, aryl, substituted aryl, benzyl, substituted benzyl, benzoyl, substituted benzoyl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl, substituted alkenyl, alkynyl, alkoxy, substituted alkoxy, acyl, halogen, amino, substituted amino, hydroxyl, hydroxylalkyl, substituted hydroxylalkyl, and —COOH.

In one embodiment, the stretch of DNA molecule comprises at least 5, 6, 7, 8, 9, 10, 11, 12, 13, 14, 15, 16, 17, 18, 19, 20, 21, 22, 23, 24, 25, 26, 27, 28, 29, or 30 nucleotides. The stretch of DNA molecule may have a maximal length of, for example, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides. In some embodiments, In one embodiment, the stretch of DNA molecule comprises, for example, 5-80, 5-75, 5-70, 10-65, 15-60, 15-55, 15-50, 15-45, 15-40, 15-35, 15-30, 15-25, 15-20, 20-60, 25-55, 25-50, 30-50, 35-45, or 30-40 nucleotides. In specific embodiments, the stretch of DNA molecule comprises 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides.

In one embodiment, the DNA molecule comprises a sequence unique from the genomes of, for example, human, mouse, and rat, comprising GC content of about 20-80%, about 30-80%, about 40-70%, about 40-60%, about 40-50%, about 50-70%, about 60-70%, or about 50-60%, and has a melting temperature of 25-55° C.

In certain embodiments, the DNA part comprises a stretch of DNA molecule comprising one or more sequences selected from SEQ ID NOs: 1-223. In some embodiments, the DNA part comprises a stretch of DNA molecule comprising one or more DNA repeats comprising one or more sequences selected from SEQ ID NOs: 1-223. In a further embodiment, the DNA part comprises a stretch of DNA molecule comprising one or more sequences selected from SEQ ID NOs: 1-53 (Table 1).

TABLE 1
sample DNA molecules

| SEQ ID | | SEQ ID | |
|---|---|---|---|
| NO: | Sequence | NO: | Sequence |
| 1 | ACCAATAATA | 2 | AATAAACCTA |
| 3 | ACATCATCAT | 4 | CCAATAATA |
| 5 | ATAAACCTA | 6 | CATCATCAT |
| 7 | CAACTTAAC | 8 | TCTAAAATC |
| 9 | AATACTCTC | 10 | TTATTCACT |
| 11 | CTTTTTTTC | 12 | CCTTCTATT |
| 13 | CTCTACTAC | 14 | TAAAAACTC |
| 15 | AACTAATCT | 16 | TTTCTCTTC |
| 17 | AACATACTA | 18 | TTCATTTAC |
| 19 | ATCCTACAA | 20 | CAATCAAAA |
| 21 | CTTACAAAC | 22 | ACAAATAAC |
| 23 | TTTTCTACC | 24 | CCCTTATTT |
| 25 | TCTTTCATT | 26 | TTCTTACTC |
| 27 | CCATAAATC | 28 | CATTTATCC |
| 29 | ATACTTCAC | 30 | TACCTCTAA |
| 31 | CTCCTATTT | 32 | CTATCCAAA |
| 33 | ATCCCTATC | 34 | TCATTACTT |
| 35 | CTAAATCTC | 36 | ACTACTTTT |
| 37 | TACTATCTC | 38 | ATATCTTCC |
| 39 | ACTAACTAT | 40 | TTATCAACT |
| 41 | TAACTTTTC | 42 | TCTTTACAT |
| 43 | CCTATACTT | 44 | TTCTTCTTT |
| 45 | TCACATAAT | 46 | ATCATATCA |
| 47 | TTTCTATCT | 48 | TCCTTTTAT |
| 49 | TCTTATACC | 50 | CATATTACA |
| 51 | TTCCTAATC | 52 | TAATCTACA |
| 53 | TAAAAATCT | | |

In preferred embodiments, the DNA part comprises a stretch of DNA molecule comprising one or more sequences selected from ACCAATAATA (SEQ ID NO: 1), AATAAACCTA (SEQ ID NO: 2), and ACATCATCAT (SEQ ID NO: 3). In a further embodiment, the DNA molecule comprises, for example, ACCAATAATA (SEQ ID NO: 1), AATAAACCTA (SEQ ID NO: 2), and/or ACATCATCAT (SEQ ID NO: 3).

In some embodiments, the DNA molecule comprises one or more repeats of ACCAATAATA (SEQ ID NO: 1), AATAAACCTA (SEQ ID NO: 2), and/or ACATCATCAT (SEQ ID NO: 3). In specific embodiments, the DNA molecule comprises -(ACCAATAATA)ₘ-, -(AATAAACCTA)ₘ-, and/or -(ACATCATCAT)ₘ-, wherein 1≤m≤10, 1≤m≤9, 1≤m≤8, 1≤m≤7, 1≤m≤6, 1≤m≤5, 1≤m≤4, or 1≤m≤3. In a preferred embodiment, the DNA molecule comprises -(ACCAATAATA)₆-, -(AATAAACCTA)₆-, and/or -(ACATCATCAT)₆-.

In a specific embodiment, the DNA part comprises a stretch of DNA molecule, of length 9-60, or 15-60 nucleotide (nt), with a sequence that is unique from the genomes of, for example, human, mouse, and rat, has GC content of 40-70%, and a melting temperature of 25-55° C.

In one embodiment, the DNA part further comprises one or more modifications in the DNA molecule. The one or more modifications may be at the 5′- end, 3′- end and/or in the backbone of the DNA molecule. In a specific embodiment, the modification is an amine group replacing the 5′- or 3′-end hydroxyl group of the DNA molecule.

In specific embodiments, the linker part comprises 1) a covalent attachment to the affinity moiety and the DNA part via amide bonds or thioether groups, and/or 2) a polyethylene glycol (PEG) linker with a total of, for example, 0-15 repeats, and/or a stretch of aliphatic carbon chains with a total of, for example, 0-16 —CH2- repeats. The linker also may comprise 1-3 sulphation groups at any position.

In specific embodiments, the linker comprises 0-15, 0-14, 0-13, 0-12, 0-11, 0-12, 0-10, 0-9, 0-8, 0-7, 0-6, 0-5, 0-4, 0-3, 1-14, 1-13, 1-12, 1-11, 1-10, 1-9, 1-8, 1-7, 1-6, 1-5, 1-4, 2-14, 2-13, 2-12, 2-11, 2-10, 2-9, 2-8, 2-7, 2-6, 2-5, 2-4, 3-14, 3-13, 3-12, 3-11, 3-10, 3-9, 3-8, 3-7, 3-6, 4-14, 4-13, 4-12, 4-11, 4-10, 4-9, 4-8, 4-7, 5-14, 5-13, 5-12, 5-11, 5-10, 5-9, 6-14, 6-13, 6-12, 6-11, 6-10, 6-9, 7-14, 7-13, 7-12, 7-11, 7-10, 7-9, 8-14, 8-13, 8-12, 8-11, 9-14, 9-13, 9-12, 10-14, 10-13, or 10-12 repeats of PEG.

In some embodiments, the linker comprises a stretch of aliphatic carbon chain with a total of, for example, 0-16, 0-15, 0-14, 0-13, 0-12, 0-11, 0-12, 0-10, 0-9, 0-8, 0-7, 0-6, 0-5, 0-4, 0-3, 1-14, 1-13, 1-12, 1-11, 1-10, 1-9, 1-8, 1-7, 1-6, 1-5, 1-4, 2-14, 2-13, 2-12, 2-11, 2-10, 2-9, 2-8, 2-7, 2-6, 2-5, 2-4, 3-14, 3-13, 3-12, 3-11, 3-10, 3-9, 3-8, 3-7, 3-6, 4-14, 4-13, 4-12, 4-11, 4-10, 4-9, 4-8, 4-7, 5-14, 5-13, 5-12, 5-11, 5-10, 5-9, 6-14, 6-13, 6-12, 6-11, 6-10, 6-9, 7-14, 7-13, 7-12, 7-11, 7-10, 7-9, 8-14, 8-13, 8-12, 8-11, 9-14, 9-13, 9-12, 10-14, 10-13, or 10-12 —CH2- repeats.

In certain embodiments, the linkage group comprises: 1) a 1,2,3-triazole linkage group formed by strain-promoted alkyne-azide cycloaddition, or copper (I)-catalyzed alkyne-azide cycloaddition; 2) an amide linkage group formed by Staudinger ligation, imidoestser-amine reaction, or a primary amine reacting with a carboxyl group activated by N-hydroxysuccinimides, tetra- or pentafluorophenol, sulfodichlorphenol, or carbodiimides; 3) a bicyclic linkage formed by cycloaddition of tetrazines and trans-cyclooctenes; and/or 4) a disulfide bond linkage.

In specific embodiments, the linkage group is: 1) a 1,2,3-triazole linkage group formed by strain-promoted alkyne-azide cycloaddition, or copper (I)-catalyzed alkyne-azide cycloaddition; 2) an amide linkage group formed by Staudinger ligation, imidoestser-amine reaction, or a primary amine reacting with a carboxyl group activated by N-hydroxysuccinimides, tetra- or pentafluorophenol, sulfodichlorphenol, or carbodiimides; 3) a bicyclic linkage formed by cycloaddition of tetrazines and trans-cyclooctenes; or 4) a disulfide bond linkage.

In a specific embodiment, the DNA-barcoding reagent according to the subject invention comprises a structure of

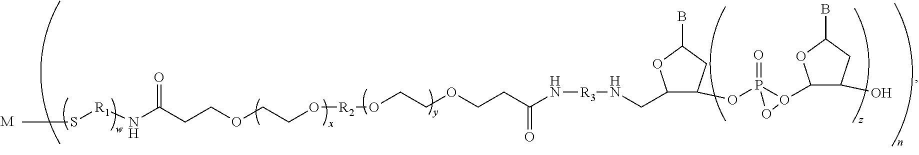

wherein M is an affinity moiety; W is 0 or 1; 0≤x+y≤15; 15≤z≤60; 1≤n≤10; R₁, R₂ and R₃ can be any chemical group; and B is selected from adenine

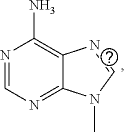

guanine

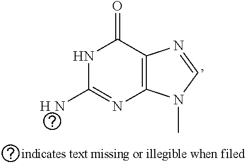

cytosine

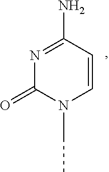

and thymine

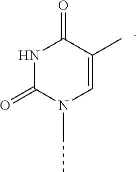

In specific embodiments, R₁, R₂ and R₃ are independently selected from, for example, O, S, alkylene, substituted alkylene, arylene, substituted arylene, heteroalkylene, substituted heteroalkylene, heteroarylene, substituted heteroarylene, cycloalkylene, substituted cycloalkylene, heterocycloalkylene, substituted heterocycloalkylene, cycloalkenylene, substituted cycloalkenylene, alkenylene, substituted alkenylene, alkynylene, substituted alkynylene, —NR₄—, —CO—, and —COO—, wherein R₄ is selected from, for example, hydrogen, alkyl, substituted alkyl, aryl, substituted aryl, benzyl, substituted benzyl, benzoyl, substituted benzoyl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl, substituted alkenyl, alkynyl, alkoxy, substituted alkoxy, acyl, halogen, amino, substituted amino, hydroxyl, hydroxylalkyl, substituted hydroxylalkyl, and —COOH.

In certain embodiments, the range of x+y may be 0≤x+y≤14, 1≤x+y≤15, 2≤x+y≤15, 3≤x+y≤15, 4≤x+y≤15, 5≤x+y≤15, 6≤x+y≤15, 7≤x+y≤15, 8≤x+y≤15, 9≤x+y≤15, 10≤x+y≤15, 11≤x+y≤15, 12≤x+y≤15, 2≤x+y≤14, 3≤x+y≤13, 4≤x+y≤12, 5≤x+y≤11, 6≤x+y≤10, or 7≤x+y≤10.

In some embodiments, 15≤z≤50; 15≤z≤40; 15≤z≤30; or 15≤z≤20. Specifically, z can be, for example, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. The range of n may be 1≤n≤9, 1≤n≤8, 1≤n≤7, 1≤n≤6, 1≤n≤5, 1≤n≤4, or 1≤n≤3.

The subject invention provides methods of using the DNA-barcoding reagent to label antibodies for producing DNA-barcoding antibodies, and methods of using the DNA-barcoding antibodies in, for example, single-cell technologies, ELISA, multiplexed immunofluorescence for histology, super-resolution imaging, and expansion microscopy.

The DNA-barcoding reagent according to the subject invention can be used for commercially available primary antibodies. In specific embodiments, the DNA-barcoding reagent comprises, consists essentially of, or consists of a secondary antibody Fab fragment or V_{HH} fragment conjugated to a DNA barcode. With this reagent, commercially available antibodies can be DNA-barcoded in a single 10-minute incubation step at room temperature, achieving quantitative yields. The so-formed complex can be used directly or after simple clean up with an additional 10-minute incubation step, for many biotechnological applications without further purification.

In one embodiment, the subject invention provides a method for producing the DNA-barcoding reagent of the subject invention, the method comprising activating an affinity moiety with a diarylcyclooctyne moiety (e.g., DBCO); activating one or more DNA oligos with azide; mixing the activated affinity moiety with the activated one or more DNA oligos to form a conjugate; optionally, removing excess of the diarylcyclooctyne moiety and/or azide; and obtaining the DNA-barcoding reagent of the subject invention.

In one embodiment, the method for producing the DNA-barcoding reagent of the subject invention further comprises purifying the DNA-barcoding reagent of the subject invention.

In specific embodiments, the affinity moiety and/or the DNA oligos may be linked to the linker, as disclosed in the subject invention, prior to the activation step. In certain embodiments, the DNA oligos may be modified with a functional group (e.g., amine) prior to the activation step.

The subject invention also provides a method for DNA-barcoding a primary antibody, comprising: providing the primary antibody; mixing and reacting the primary antibody with the DNA-barcoding reagent of the subject invention for, e.g., 10 min to conjugate the DNA-barcoding reagent with the antibody, and collecting the DNA-barcoded antibody, wherein collecting the DNA-barcoded antibody may comprise adding non-specific IgG to the mixture of the primary antibody and the DNA-barcoding reagent to scavenge excess reagent.

In certain embodiments, the method for DNA-barcoding an antibody does not require or need the activation of the primary antibody and DNA with reactive groups, reacting the activated primary antibody and DNA in a conjugation reaction, and purifying and concentrating the product.

In a specific embodiment, the DNA-barcoded antibody according to the subject invention comprises a structure of

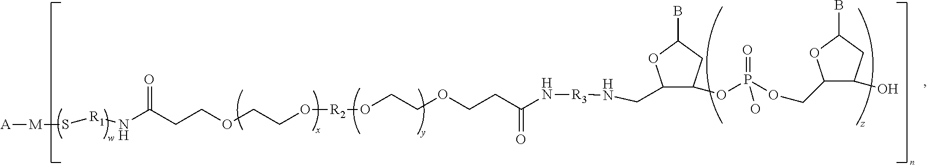

wherein A is an antibody; M is an affinity moiety; W is 0 or 1; 0≤x+y≤15; 15≤z≤60; 1≤n≤10; R₁, R₂ and R₃ can be any chemical group; and B is selected from adenine

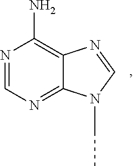

guanine

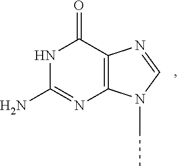

cytosine

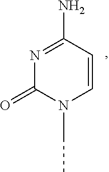

and thymine

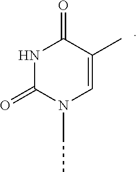

In certain embodiments, the range of x+y may be 0≤x+y≤14, 1≤x+y≤15, 2≤x+y≤15, 3≤x+y≤15, 4≤x+y≤15, 5≤x+y≤15, 6≤x+y≤15, 7≤x+y≤15, 8≤x+y≤15, 9≤x+y≤15, 10≤x+y≤15, 11≤x+y≤15, 12≤x+y≤15, 2≤x+y≤14, 3≤x+y≤13, 4≤x+y≤12, 5≤x+y≤11, 6≤x+y≤10, or 7≤x+y≤10.

In some embodiments, 15≤z≤50; 15≤z≤40; 15≤z≤30; or 15≤z≤20. Specifically, z can be, for example, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. The range of n may be 1≤n≤9, 1≤n≤8, 1≤n≤7, 1≤n≤6, 1≤n≤5, 1≤n≤4, or 1≤n≤3. Preferably, 1≤n≤3.

In specific embodiments, R₁, R₂ and R₃ are independently selected from, for example, O, S, alkylene, substituted alkylene, arylene, substituted arylene, heteroalkylene, substituted heteroalkylene, heteroarylene, substituted heteroarylene, cycloalkylene, substituted cycloalkylene, heterocycloalkylene, substituted heterocycloalkylene, cycloalkenylene, substituted cycloalkenylene, alkenylene, substituted alkenylene, alkynylene, substituted alkynylene, —NR₄—, —CO—, and —COO—, wherein R₄ is selected from, for example, hydrogen, alkyl, substituted alkyl, aryl, substituted aryl, benzoyl, substituted benzoyl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl, substituted alkenyl, alkynyl, alkoxy, substituted alkoxy, acyl, halogen, amino, substituted amino, hydroxyl, hydroxylalkyl, substituted hydroxylalkyl, and —COOH.

As used herein, “alkyl” means linear saturated monovalent radicals of at least one carbon atom or a branched saturated monovalent of at least three carbon atoms. It may include hydrocarbon radicals of at least one carbon atom, which may be linear. It includes, for example, C1-C10 alkyl. Examples include, but not limited to, methyl, ethyl, propyl, 2-propyl, n-butyl, iso-butyl, tert-butyl, pentyl, hexyl, and the like.

As used herein, “alkylene” means linear saturated divalent radicals of at least one carbon atom or a branched saturated divalent of at least three carbon atoms. It may include alkanediyl group. It may also include hydrocarbon radicals of at least one carbon atom, which may be linear. It includes, for example, C1-C10 alkylene. Examples include, but not limited to, methylene, ethylene, propylene, 2-propyl, butylene, pentylene, hexylene, and the like. The free valencies may or may not be on adjacent carbon atoms.

As used herein, “acyl” means a radical —C(O)R where R includes, but is not limited to, hydrogen, alkyl, aryl, benzyl, benzoyl, heteroalkyl, heteroaryl, cycloalkyl, heterocycloalkyl, cycloalkenyl, alkenyl, alkynyl, alkoxy, sulfhydryl, halogen, amino, hydroxyl, hydroxylalkyl. Examples include, but are not limited to, formyl, acetyl, ethylcarbonyl, and the like. An aryl group may be substituted or unsubstituted.

As used herein, “alkenyl” refers to a straight or branched hydrocarbon chain containing one or more double bonds. The alkenyl group may have 2 to 9 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C2-4 alkenyl” or similar designations. By way of example only, “C2-4 alkenyl” indicates that there are two to four carbon atoms in the alkenyl chain, i.e., the alkenyl chain is selected from ethenyl; propen-1-yl; propen-2-yl; propen-3-yl; buten-1-yl; buten-2-yl; buten-3-yl; buten-4-yl; 1-methyl-propen-1-yl; 2-methyl-propen-1-yl; 1-ethyl-ethen-1-yl; 2-methyl-propen-3-yl; buta-1,3-dienyl; buta-1,2,-dienyl and buta-1,2-dien-4-yl. Typical alkenyl groups include, but are in no way limited to, ethenyl, propenyl, butenyl, pentenyl, and hexenyl, and the like.

As used herein, “alkenylene” refers to straight or branched divalent radicals of hydrocarbon chain containing one or more double bonds. The alkenylene group may have, for example, 2 to 9 carbon atoms, although the present definition also covers the occurrence of the term “alkenylene” where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C2-4 alkenylene” or similar designations. Typical alkenyl groups include, but are in no way limited to, ethenylene, propenylene, butenylene, pentenylene, and hexenylene, and the like.

As used herein, “alkynyl” refers to a straight or branched hydrocarbon chain comprising one or more triple bonds. The alkynyl group may have 2 to 9 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C2-4 alkynyl” or similar designations. By way of example only, “C2-4 alkynyl” indicates that there are two to four carbon atoms in the alkynyl chain, e.g., the alkynyl chain is selected from ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “alkynylene” refers to straight or branched divalent radicals of hydrocarbon chain comprising one or more triple bonds. The alkynylene group may have 2 to 9 carbon atoms, although the present definition also covers the occurrence of the term “alkynylene” where no numerical range is designated. The alkynylene group may also be a medium size alkynylene having 2 to 9 carbon atoms. The alkynylene group could also be a lower alkynylene having 2 to 4 carbon atoms. The alkynylene group may be designated as “C2-4 alkynylene” or similar designations. Typical alkynylene groups include, but are in no way limited to, ethynylene, propynylene, butynylene, pentynylene, and hexynylene, and the like.

As used herein, “cycloalkyl” means a fully saturated carbocyclyl ring or ring system. Examples include, but are not limited to, cyclopropyl, cyclobutyl, cyclopentyl, and cyclohexyl.

As used herein, “cycloalkylene” means fully saturated divalent radicals of carbocyclyl ring or ring system. Examples include, but are not limited to, cyclopropylene, cyclobutylene, cyclopentylene, and cyclohexylene.

As used herein, “aryl” refers to a carbocyclic (all carbon) monocyclic or multicyclic aromatic ring system (including fused ring systems where two carbocyclic rings share a chemical bond). The number of carbon atoms in an aryl group can vary. For example, the aryl group can be a C6-C14 aryl group, a C6-C10 aryl group, or a C6 aryl group. Examples of aryl groups include, but are not limited to, phenyl, benzyl, α-naphthyl, β-naphthyl, biphenyl, anthryl, tetrahydronaphthyl, fluorenyl, indanyl, biphenylenyl, and acenaphthenyl. Preferred aryl groups are phenyl and naphthyl.

As used herein, “arylene” refers to a divalent radical of carbocyclic (all carbon) monocyclic or multicyclic aromatic ring system (including fused ring systems where two carbocyclic rings share a chemical bond). The number of carbon atoms in an arylene group can vary. For example, the arylene group can be a C6-C14 arylene group, a C6-C10 arylene group, or a C6 arylene group.

As used herein, “heteroaryl” refers to an aromatic ring or ring system (i.e., two or more fused rings that share two adjacent atoms) that comprise(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

As used herein, “heteroarylene” refers to a divalent radical of aromatic ring or ring system (i.e., two or more fused rings that share two adjacent atoms) that comprise(s) one or more heteroatoms, that is, an element other than carbon, including but not limited to, nitrogen, oxygen and sulfur, in the ring backbone. When the heteroarylene is a ring system, every ring in the system is aromatic. The heteroarylene group may have 5-18 ring members (i.e., the number of atoms making up the ring backbone, including carbon atoms and heteroatoms), although the present definition also covers the occurrence of the term “heteroarylene” where no numerical range is designated.

As used herein, “halogen” refers to an atom of fluorine, chlorine, bromine or iodine.

As used herein, a “substituted” group may be substituted with one or more group(s) individually and independently selected from alkyl, alkenyl, alkynyl, cycloalkyl, cycloalkenyl, cycloalkynyl, benzyl, substituted benzyl, substituted benzoyl, benzoyl, acetyl, aryl, heteroaryl, heteroalicyclyl, aralkyl, heteroaralkyl, (heteroalicyclyl)alkyl, hydroxy, protected hydroxyl, alkoxy, aryloxy, acyl, carboxyl, COOR, CH₂COR, CH₂COOR, mercapto, alkylthio, arylthio, cyano, halogen, thiol, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group, alkylamino, arylamino, amino acid(s) and protected derivatives thereof.

In one embodiment, the subject invention provides a composition comprising the DNA-barcoding reagent or the DNA-barcoded antibody of the subject invention. The composition further comprises a pharmaceutically acceptable carrier

“Pharmaceutically acceptable carrier” or “pharmaceutically acceptable excipient” includes any and all solvents, dispersion media, coatings, antibacterial and antifungal agents, isotonic, and absorption delaying agents, and the like. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions.

In one embodiment, the subject invention provides a method for multiplexed molecular (e.g., antigen) imaging and profiling in a sample (e.g., a tissue sample), comprising contacting one or more DNA-barcoded antibodies with the sample, wherein each DNA-barcoded antibody comprising an affinity moiety specific to an antibody, wherein the antibody is covalently linked to one or more DNA sequences or via a linker, and one or more DNA sequences are directly or indirectly labelled with one or more reporters, such as fluorophores and wherein each antibody targets each molecule (e.g., antigen) of interest.

With the use of DNA-barcoded antibodies, multiplexed tissue antigen imaging can help clarify interrelationships between different molecules and cells in terms of their distributions. In multiplexed immunoassays, these methods will allow the quantification of many proteins at once. In single-cell technologies, multiplexed sorting of individual cells with certain markers can be aided by flow cytometry and the use of DNA-barcoded antibodies.

Advantageously, the method can detect 1-100 antigens in the same sample, preferably, a tissue sample. The method provides images using fluorescent microscope techniques (e.g. but not limited to confocal microscopy, two- or multi-photon microscopy, light sheet microscopy, super-resolution microscopy techniques), with or without augmentation by tissue clearing techniques.

In some embodiments, the reporters labeling the DNA sequence may be attached to either the 5′ or 3′ end of the sequence. The label may also be attached with the backbone of the sequence. The skilled person is aware of techniques for attaching labels to nucleic acid strands. If the label is indirectly attached to the nucleic acid sequence, it may be by any mechanism known to one of skill in the art, such as using biotin and streptavidin, and a complementary sequence. In specific embodiments, the DNA sequence may bind to a complementary sequence that is labeled with the reporter.

In certain embodiments, the reporter label may include, but is not limited to a fluorescent dye, an enzyme, an organic donor fluorophore or an organic acceptor fluorophore, a luminescent lanthanide, a fluorescent or luminescent nanoparticle, or an affinity tag such as biotin. In specific embodiments, the fluorescent label may be, for example, fluorescein, TAMRA, rhodamine, Texas Red, AlexaFluor (e.g., AlexaFluor 488, AlexaFluor 532, AlexaFluor 546, AlexaFluor 594, AlexaFluor 633 and AlexaFluor 647), cyanine dye (e.g., Cy7, Cy7.5, Cy5, Cy5.5 and Cy3), Tye dye (e.g., TYE 563, TYE 665, TYE 705), atto dye (e.g., Atto 594 and Atto 633), Hexachlorofluorescein, FAM (6-carboxyfluroescein), BODIPY FL, OliGreen, 40,6-diamidino-2-phenylindol (DAPI), Hoechst 33,258, malachite green (MG), and FITC. In a further embodiment, the fluorophore is selected from the group consisting of fluorophores that emit a blue, green, or red (e.g., near red or far red) fluorescence.

In one embodiment, the sample may be a biological sample of a subject. In specific embodiments, the biological sample may be, for example, tissue, blood, or plasma. The biological sample may also be primary cells or cultured cells, e.g., adherent cells or cells in suspension. “Subject” refers to an animal, such as a mammal, for example a human. The methods described herein can be useful in both pre-clinical human therapeutics and veterinary applications. In some embodiments, the subject is a mammal (such as an animal model of disease), and in some embodiments, the subject is human. Non-limiting examples of subjects include canine, porcine, rodent, feline, bovine, poultry, equine, human, and a non-human primate.

In one embodiment, the subject invention provides a method for revealing/detecting the distribution of one or more biomolecules in a sample, preferably, a tissue sample, the method comprising contacting one or more DNA-barcoded antibodies with the sample. The biomolecule may be, for example, protein, fragment thereof, or polypeptide. Each antibody targets each biomolecule of interest.

In one embodiment, the subject invention provides a method for detecting one or more biomolecules in a sample, preferably, a tissue sample, the method comprising contacting one or more DNA-barcoded antibodies with the sample.

In a further embodiment, the presence of one or more biomolecules in the sample may be indicated by the reporter labeled with the DNA sequence of the DNA-barcoding reagent. The one or more biomolecules is detected using fluorescent microscope techniques, e.g. confocal microscopy, two- or multi-photon microscopy, light sheet microscopy, and super-resolution microscopy techniques.

In one embodiment, the method for detecting one or more biomolecules in the sample may involve the use of complementary fluorescent-labeled DNA oligos that recognize or hybridize with the DNA sequence of the DNA-barcoding reagent.

In one embodiment, the method may further comprise measuring the concentration of the biomolecule in the sample by comparing the signal, e.g., fluorescence intensity, from the reporter labeled with the DNA sequence or the complementary fluorescent-labeled DNA oligo to a standard curve of such label.

In one embodiment, the complementary DNA oligo is selected from sequences that are complementary to SEQ ID NOs: 1-223.

In one embodiment, the subject invention provides a method for multiplexed biomolecule detection in the form of an enzyme-linked immunosorbent assay or assays of a similar principle by using the DNA-barcoded antibodies according to the subject invention. The method comprises contacting one or more DNA-barcoded antibodies with a sample.

In one embodiment, the subject invention provides a method for identifying cells that express a biomarker in a sample, the method comprising contacting the DNA-barcoded antibody with the sample, wherein the antibody is specific for the biomarker; and identifying cells that express the biomarker of interest.

In one embodiment, the subject invention provides a method for quantifying cells that express a biomarker in a sample, the method comprising contacting the DNA-barcoded antibody with the sample, wherein the antibody is specific for the biomarker; and quantifying cells that express the biomarker of interest.

In one embodiment, the subject invention provides a method for isolating cells that express a biomarker in a sample, the method comprising contacting the DNA-barcoded antibody with the sample, wherein the antibody is specific for the biomarker; identifying cells that express the biomarker of interest; and isolating cells that express the biomarker of insterest.

In one embodiment, the subject invention provides a method for affinity purification and sorting of marker-positive cells by, for example, flow cytometry and DNA column chromatography. The method comprises contacting one or more DNA-barcoded antibodies with a sample; identifying marker-positive cells; and purifying or sorting the marker-positive cells.

In one embodiment, the method of the subject invention may further comprise contacting/mixing one or more complementary fluorescent-labeled DNA oligos that recognize or hybridize with the DNA sequence of the DNA-barcoding reagent with the sample.

In one embodiment, the subject invention provides a method for the purpose of expansion microscopy. The method comprises contacting one or more DNA-barcoded antibodies with a sample.

In one embodiment, the subject invention provides a method for parallel protein purification with the aid of DNA arrays. The method comprises contacting one or more DNA-barcoded antibodies with a sample.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” and “comprise” can be used interchangeably; “consisting essentially of,” and “consists essentially of” can be used interchangeably; and “consisting,” and “consists” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting essentially of” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Where particular values are described in the application and claims, unless otherwise stated the term “about” meaning within an acceptable error range for the particular value should be assumed.

### MATERIALS AND METHODS

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1

### Comparison with Existing or Previous Product

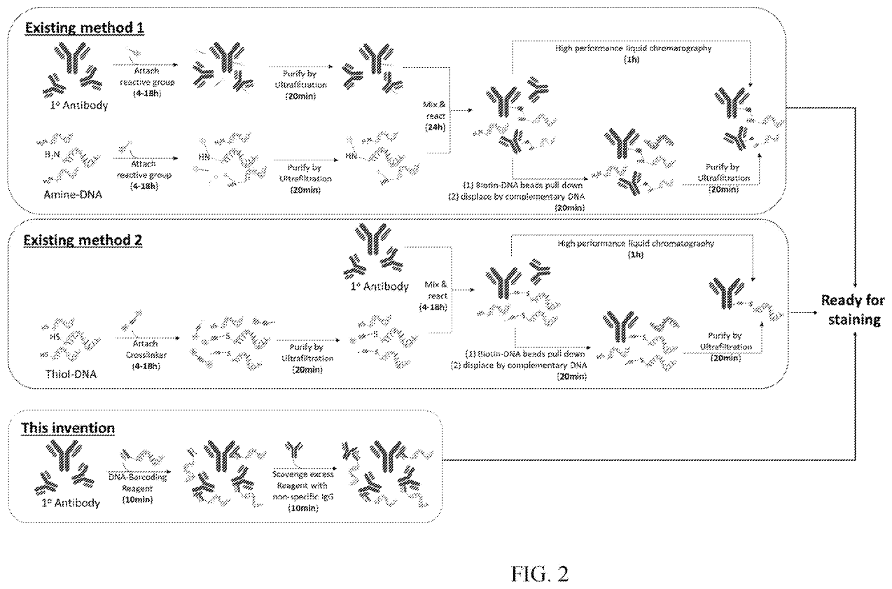

The present invention aligns with existing multiplexing methods using DNA-barcoded antibodies to label or detect biomolecules (FIG. 2). These methods of conjugating DNAs to antibodies for a multitude of applications invariably involve bioconjugation chemical reactions. Due to the low reaction efficiency and specificity, tedious preparation and large-scale reactions are required to generate minute quantities of the DNA-conjugated antibodies.

Cremers et al. presented a concept similar to the instant DNA-barcoding reagent. Their affinity moiety consists of Protein G—a protein from Streptococcus spp that binds specifically to a certain region in antibodies. The instant approach is superior in several ways:

(1) The size of Protein G is 21.8 kDa, whereas that of Fab fragment is 50 kDa (inferior) and V_{HH} fragment is 14 kDa (superior);

(2) The instant method is generalizable to Chicken IgY as well as immunoglobulins raised in other species, whereas a similarly high-affinity Protein G mutant targeting these immunoglobulins may not be easily generated;

(3) The Fab fragments (and increasingly, V_{HH} fragments) are shown to be compatible with a range of biological applications, including histology, super-resolution imaging, flow cytometry, sequencing applications, etc, whereas a bacterial protein may interfere with these assays; and

(4) their barcoding reaction requires 1 hour of incubation at 4° C., whereas the instant incubation only requires 10 minutes at room temperatures.

The instant invention is based on the combination of the DNA-barcoding antibody concept and the use of monovalent antibody-specific binding proteins. The outcomes are the same as existing methods, where the desired DNA sequence labels the antigens via the primary antibody. However, the instant disclosure describes a better preparation method to prepare DNA-barcoded primary antibodies in the form of a marketable reagent.

### EXAMPLE 2

### DNA Conjugated Fab Fragment of Secondary Antibody

The synthesis of DNA conjugated Fab fragment of secondary antibody occurs in two steps).

Donkey anti-Mouse secondary antibody Fab fragment unconjugated (at concentration of 1 mg/ml) was acquired from Jackson Immunoreserach (Cat no 715-007-003). To produce DBCO-labelled Fab fragments, 1 ul of the 1 mg/ml Fab fragment solution was reacted with 0.6 ul of 10 mM DBCO-PEG5-TFP (Click Chemistry Tools 1260-10) stock solution in anhydrous DMSO. The reaction was proceeded for 24 hours at room temperature with gentle agitation. The DBCO-labelled Fab fragments were then purified and concentrated with Ultracentrifugal units (Millipore UFC501024), diluted to 1 mg/ml in concentration and stored at 4° C. until use.

Amine-modified oligos with DNA structures 5′-NH₂-(ACCAATAATA)₆, 5′-NH₂-(AATAAACCTA)₆, and 5′-NH₂-(ACATCATCAT)₆ were ordered from Integrated DNA Technologies with desalting purification. To produce azide-labelled oligos, the amine-modified oligos were reconstituted in double distilled water at 500 μM concentration. Then, 18 ul of the 500 μM amine-modified oligo stock was mixed with 2.5 ul 10×PBS and 4.5 ul 10 mM Azido-PEG4-NHS (AZ103-25) stock solution in anhydrous DMSO. The reaction was proceeded for 24 hours at room temperature with gentle agitation. The Azide-labelled oligos were purified and concentrated using spin columns (Zymo Research D7010), diluted to 200 μM and stored at −20° C. before use.

To produce DNA-conjugated Fab fragments, 30 ul of 1 mg/ml DBCO-labelled Fab fragment was reacted with 15 ul of 200 μM of an Azide-labelled oligo. The reaction was proceeded at room temperature for at least 24 hours. The DNA-conjugated Fab fragments were then purified and concentrated with Ultracentrifugal units (Millipore UFC501024), diluted to 1 mg/ml in concentration and stored at 4° C. until use.

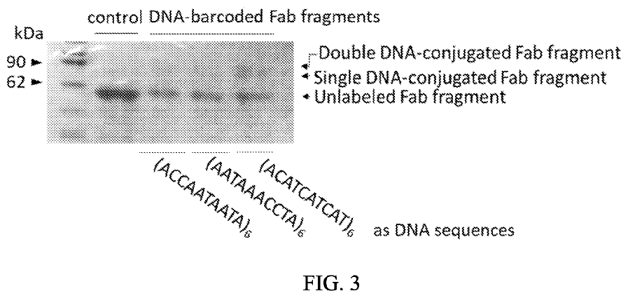

Specifically, single DNA-conjugated Fab fragments and double DNA-conjugated Fab fragments can be distinguished from the unlabeled Fab fragment. The SDS-PAGE gel electrophoresis was performed to demonstrate that DNA can be successful conjugated to Fab fragments of secondary antibodies (FIG. 3).

### EXAMPLE 3

### Multiplexed Molecular Imaging Using DNA-Barcoded Antibodies

DNA-barcoding reagents such as DNA1-Fab anti Ms, DNA2-Fab anti Ms, DNA3-Fab anti Ms were synthesized as in EXAMPLE 2. The sequences for DNA1, DNA2, and DNA3 are (ACCAATAATA)₆, (AATAAACCTA)₆, and (ACATCATCAT)₆, respectively. DNA-barcoding reagents such as DNA1-Fab anti-Ms, DNA2-Fab anti-Ms, DNA3-Fab anti-Ms are complexed with Ms anti-CD56, anti-CD3 and anti-CD19, respectively, to form Ms anti-CD56/DNA1-Fab anti-Ms complex, Ms anti-CD3/DNA2-Fab anti-Ms complex, and Ms anti-CD19/DNA3-Fab anti-Ms complex by mixing 1 μg of the DNA-barcoding reagent with the 1 μg primary antibody in 10 μl 1×PBS for 10 minutes at room temperature. Buffers such as PBS (1×PBS), PBST (0.1% Triton X-100 in 1×PBS), PBSTB (1×PBS with 2% w/v BSA and 0.1% v/v Triton X-100) and Staining buffer (1×PBS with 2% w/v BSA, 0.1% v/v Triton X-100, 0.2 mg/ml sheared salmon sperm, and 4 mM EDTA) were prepared.

These DNA-barcoded antibodies are used to stain a human oral squamous cell carcinoma sample. The human oral squamous cell carcinoma was fixed in 10% neutral buffered formalin at room temperature for 4 hours, washed 2 times for 10 minutes in PBS, washed 3 times for 20 minutes PBSTB, washed 2 times for 5 minutes in PBS before proceeding to staining. 1 μg of each of Ms anti-CD56/DNA1-Fab anti-Ms complex, Ms anti-CD3/DNA2-Fab anti-Ms complex, and Ms anti-CD19/DNA3-Fab anti-Ms complex were added to 200 μl of Staining buffer, which were then added to the tissue and incubated for 4 hours at room temperature with gentle agitation. The tissue was then washed 3 times for 10 minutes in PBSTB, and washed 2 times for 5 minutes in PBS. The tissue was then fixed in 4% paraformaldehyde in PBS for 30 minutes in room temperature. The tissue was then washed 2 times for 5 minutes in PBS, washed 1 time for 5 minutes in 100 mM NH₄Cl in 1×PBS, washed 2 times for 5 minutes in PBS and 1 time for 5 minutes in PBST.

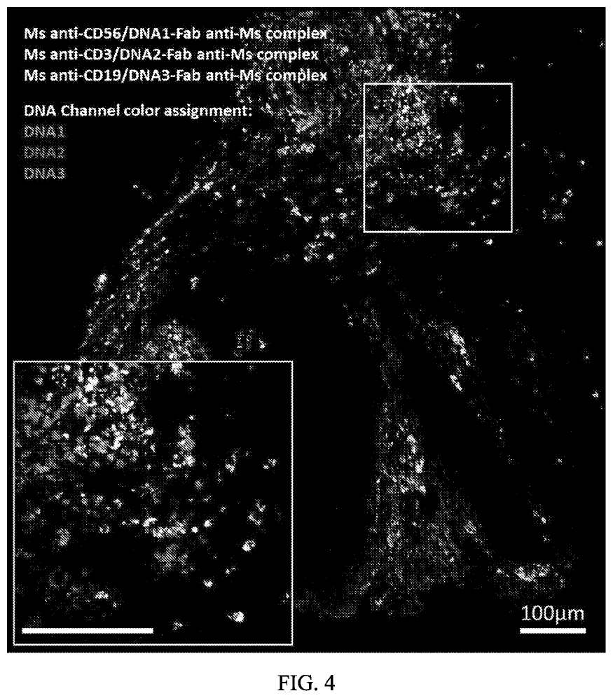

Imaging of the DNAs in the stained tissue was performed using complementary fluorescent-labeled DNA oligos, with structures consisting of 5′-FAM-TTTATTATTGGTTATTATTGGT (SEQ ID NO: 224), 5′-TexasRed-TTTAGGTTTATTTAGGTTTATT (SEQ ID NO: 225), and 5′-Cy5.5-TTATGATGATGTATGATGATGT (SEQ ID NO: 226), all ordered from General Biosystems and reconstituted in 100 μl of distilled water. The stained tissue was finally incubated in PBST with 1 μM of each fluorescent-labelled DNA oligos and incubated at room temperature. The tissue was then washed in PBST at 37° C. for 2 times 5 minutes, washed in PBS at room temperature for 10 minutes, and imaged using a confocal microscope. The tissue Multiplexed tissue antigen imaging shows the distributions of CD56, CD3 and CD19 in this sample (FIG. 4).

### EXEMPLARY EMBODIMENTS

Embodiment 1. A DNA-barcoding reagent comprising an affinity moiety, a DNA part, and an intervening linker part.

Embodiment 2. The DNA-barcoding reagent of embodiment 1, wherein the affinity moiety is a Fab fragment of an antibody originated from goat or donkey, targeting specific immunoglobulin G (IgG) class molecules from mouse, rat, rabbit, goat, or guinea pig; and immunoglobulin Y (IgY) molecules from chicken.

Embodiment 3. The DNA-barcoding reagent of embodiment 1, wherein the affinity moiety is a modified V_{HH} domain of an antibody originated from Camelidae, targeting specific immunoglobulin G (IgG) class molecules from mouse and rabbit.

Embodiment 4. The DNA-barcoding reagent of embodiment 1, wherein the DNA part comprises one or more deoxyribonucleic acid (DNA) molecules that are unique from the genomes of human, mouse, or rat.

Embodiment 5. The DNA-barcoding reagent of embodiment 4, wherein the DNA molecule comprises 15-60 nucleotides.

Embodiment 6. The DNA-barcoding reagent of embodiment 4, wherein the DNA molecule has GC content of 40-70%.

Embodiment 7. The DNA-barcoding reagent of embodiment 4, wherein the DNA molecule has a melting temperature of 25-55° C.

Embodiment 8. The DNA-barcoding reagent of embodiment 1, wherein the DNA part comprises an amine group replacing the 5′- or 3′-end hydroxyl group of one or more DNA molecules.

Embodiment 9. The DNA-barcoding reagent of embodiment 1, wherein the linker part comprises a 1,2,3-triazole linkage group formed by strain-promoted alkyne-azide cycloaddition, or copper (I)-catalyzed alkyne-azide cycloaddition.

Embodiment 10. The DNA-barcoding reagent of embodiment 1, wherein the linker part comprises a an amide linkage group formed by Staudinger ligation, imidoestser-amine reaction, or a primary amine reacting with a carboxyl group activated by N-hydroxysuccinimides, tetra- or pentafluorophenol, sulfodichlorphenol, or carbodiimides.

Embodiment 11. The DNA-barcoding reagent of embodiment 1, wherein the linker part comprises a bicyclic linkage formed by cycloaddition of tetrazines and trans-cyclooctenes.

Embodiment 12. The DNA-barcoding reagent of embodiment 1, wherein the linker part comprises a disulfide bond linkage.

Embodiment 13. The DNA-barcoding reagent of embodiment 1, which has a structure of

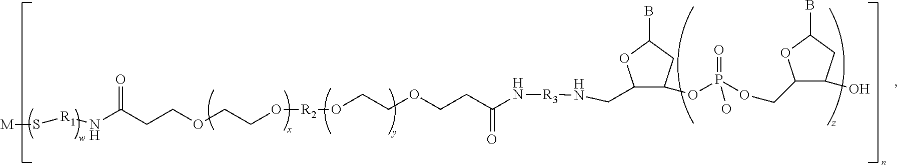

wherein M is an affinity moiety; W is 0 or 1; 0≤x+y≤15; 15≤z≤30; 1≤n≤3; B is selected from

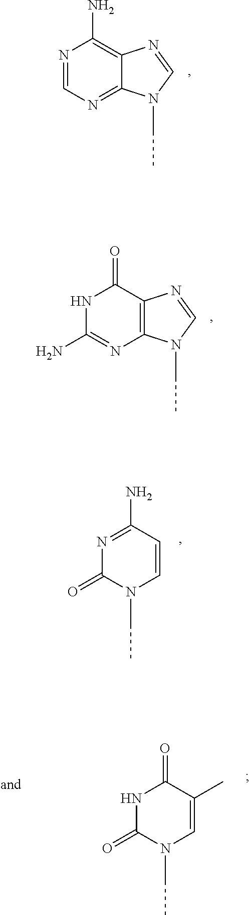

and R₁, R₂ and R₃ are independently selected from alkylene, substituted alkylene, arylene, aubstituted arylene, alkenylene, substituted alkenylene, alkynylene, substituted alkynylene, heteroalkylene, substituted heteroalkylene, heteroarylene, substituted heteroarylene, cycloalkylene, substituted cycloalkylene, heterocycloalkylene, substituted heterocycloalkylene, cycloalkenyl, —C(O)—, and —COO—.

Embodiment 14. A DNA-barcoded antibody, comprising the DNA-barcoding reagent of embodiment 1 conjugated to an antibody.

Embodiment 15. The DNA-barcoded antibody of embodiment 14, which has a structure of

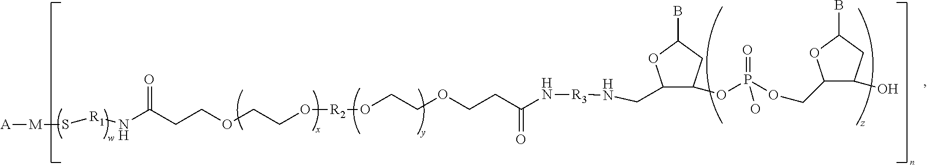

wherein A is an antibody; M is an affinity moiety; W is 0 or 1; 0≤x+y≤15; 15≤z≤30; 1≤n≤3; B is selected from

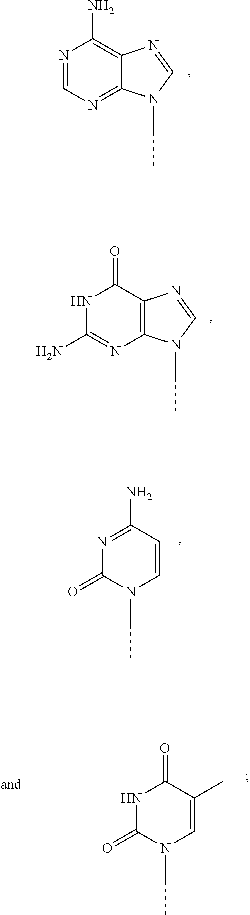

and R₁, R₂ and R₃ are independently selected from alkylene, substituted alkylene, arylene, aubstituted arylene, alkenylene, substituted alkenylene, alkynylene, substituted alkynylene, heteroalkylene, substituted heteroalkylene, heteroarylene, substituted heteroarylene, cycloalkylene, substituted cycloalkylene, heterocycloalkylene, substituted heterocycloalkylene, cycloalkenyl, —C(O)—, and —COO—.

Embodiment 16. A method for multiplexed antigen imaging and profiling in a sample, the method comprising contacting one or more DNA-barcoded antibodies of embodiment 14 with the sample, and imaging the multiplexed antigens in the sample.

Embodiment 17. The method for multiplexed antigen imaging and profiling of embodiment 16, wherein imaging the multiplexed antigens in the sample uses fluorescent microscope techniques selected from confocal microscopy, two- or multi-photon microscopy, light sheet microscopy, and super-resolution microscopy techniques.

Embodiment 18. The method for multiplexed antigen imaging and profiling of embodiment 16, wherein the step of contacting takes 10 minutes at room temperature.

Embodiment 19. A method for multiplexed biomolecule detection in a sample, the method comprising contacting one or more DNA-barcoded antibodies of embodiment 14 with the sample, and imaging the multiplexed biomolecules in the sample.

Embodiment 20. A method for identifying cells that express a biomarker of interest in a sample, the method comprising contacting the DNA-barcoded antibody of embodiment 14 with the sample, and identifying cells that express the biomarker of interest.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Dezfouli, M., Vickovic, S., Iglesias, M. J., Schwenk, J. M. & Ahmadian, A. Parallel barcoding of antibodies for DNA-assisted proteomics. PROTEOMICS 14, 2432-2436 (2014).
2. Saka, S. K. et al. Immuno-SABER enables highly multiplexed and amplified protein imaging in tissues. Nature Biotechnology 37, 1080-1090 (2019).
3. Maerle, A. v. et al. Development of the covalent antibody-DNA conjugates technology for detection of IgE and IgM antibodies by immuno-PCR. PLOS ONE 14, e0209860 (2019).
4. Sano, T., Smith, C. L. & Cantor, C. R. Immuno-PCR: Very sensitive antigen detection by means of specific antibody-DNA conjugates. Science 258, 120-122 (1992).
5. Kazane, S. A. et al. Site-specific DNA-antibody conjugates for specific and sensitive immuno-PCR. Proceedings of the National Academy of Sciences of the United States of America 109, 3731-3736 (2012).
6. van Buggenum, J. A. G. L. et al. A covalent and cleavable antibody-DNA conjugation strategy for sensitive protein detection via immuno-PCR. Scientific Reports 6, 1-12 (2016).
7. Wiener, J., Kokotek, D., Rosowski, S., Lickert, H. & Meier, M. Preparation of single- and double-oligonucleotide antibody conjugates and their application for protein analytics. Scientific Reports 10, 1-11 (2020).
8. Gong, H. et al. Simple Method to Prepare Oligonucleotide-Conjugated Antibodies and Its Application in Multiplex Protein Detection in Single Cells. Bioconjugate Chemistry 27, 217-225 (2016).
9. Cremers, G. A. O., Rosier, B. J. H. M., Riera Brillas, R., Albertazzi, L. & de Greef, T. F. A. Efficient small-scale conjugation of DNA to primary antibodies for multiplexed cellular targeting. Bioconjugate Chemistry 30, 2384-2392 (2019).
10. Ullal, A. v. & Weissleder, R. Photocleavable dna barcoding antibodies for multiplexed protein analysis in single cells. in Methods in Molecular Biology vol. 1346 47-54 (Humana Press Inc., 2015).

1. A DNA-barcoding reagent comprising an affinity moiety, a DNA part, and an intervening linker part.

2. The DNA-barcoding reagent of claim 1, wherein the affinity moiety is a Fab fragment of an antibody originated from goat or donkey, targeting specific immunoglobulin G (IgG) class molecules from mouse, rat, rabbit, goat, or guinea pig; and immunoglobulin Y (IgY) molecules from chicken.

3. The DNA-barcoding reagent of claim 1, wherein the affinity moiety is a modified V_{HH} domain of an antibody originated from Camelidae, targeting specific immunoglobulin G (IgG) class molecules from mouse and rabbit.

4. The DNA-barcoding reagent of claim 1, wherein the DNA part comprises one or more deoxyribonucleic acid (DNA) molecules that are unique from the genomes of human, mouse, or rat.

5. The DNA-barcoding reagent of claim 4, wherein the DNA molecule comprises 15-60 nucleotides.

6. The DNA-barcoding reagent of claim 4, wherein the DNA molecule has GC content of 40-70%.

7. The DNA-barcoding reagent of claim 4, wherein the DNA molecule has a melting temperature of 25-55° C.

8. The DNA-barcoding reagent of claim 1, wherein the DNA part comprises an amine group replacing the 5′- or 3′-end hydroxyl group of one or more DNA molecules.

9. The DNA-barcoding reagent of claim 1, wherein the linker part comprises a 1,2,3-triazole linkage group formed by strain-promoted alkyne-azide cycloaddition, or copper (I)-catalyzed alkyne-azide cycloaddition.

10. The DNA-barcoding reagent of claim 1, wherein the linker part comprises an amide linkage group formed by Staudinger ligation, imidoestser-amine reaction, or a primary amine reacting with a carboxyl group activated by N-hydroxysuccinimides, tetra- or pentafluorophenol, sulfodichlorphenol, or carbodiimides.

11. The DNA-barcoding reagent of claim 1, wherein the linker part comprises a bicyclic linkage formed by cycloaddition of tetrazines and trans-cyclooctenes.

12. The DNA-barcoding reagent of claim 1, wherein the linker part comprises a disulfide bond linkage.

13. The DNA-barcoding reagent of claim 1, which has a structure of 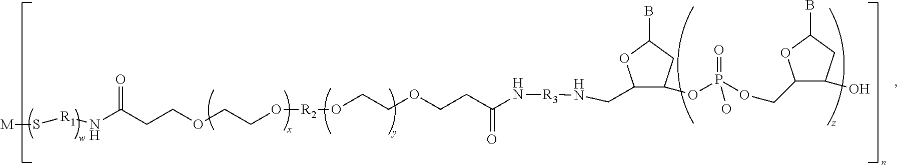
wherein M is an affinity moiety; W is 0 or 1; 0≤x+y≤15; 15≤z≤30; 1≤n≤3; B is selected from 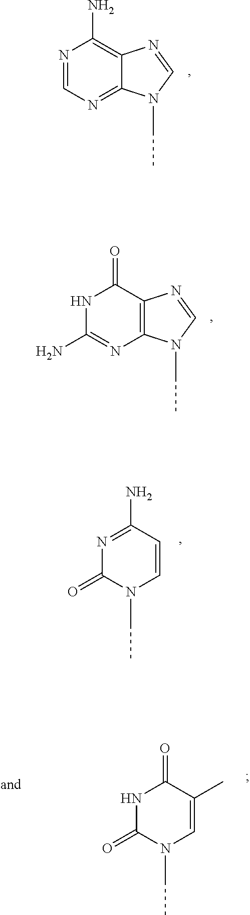
and R₁, R₂ and R₃ are independently selected from alkylene, substituted alkylene, arylene, substituted arylene, alkenylene, substituted alkenylene, alkynylene, substituted alkynylene, heteroalkylene, substituted heteroalkylene, heteroarylene, substituted heteroarylene, cycloalkylene, substituted cycloalkylene, heterocycloalkylene, substituted heterocycloalkylene, cycloalkenyl, —C(O)—, and —COO—.

14. A DNA-barcoded antibody, comprising the DNA-barcoding reagent of claim 1 conjugated to an antibody.

15. The DNA-barcoded antibody of claim 14, which has a structure of 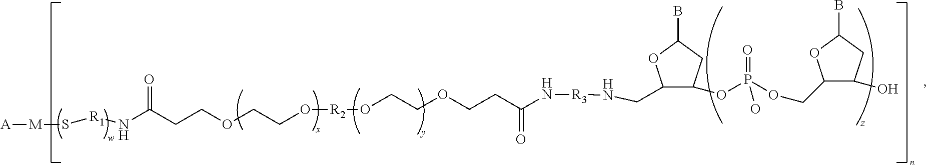
wherein A is an antibody; M is an affinity moiety; W is 0 or 1; 0≤x+y≤15; 15≤z≤30; 1≤n≤3; B is selected from 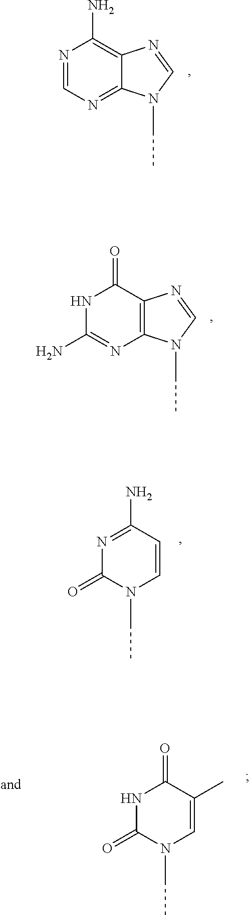
and R₁, R₂ and R₃ are independently selected from alkylene, substituted alkylene, arylene, substituted arylene, alkenylene, substituted alkenylene, alkynylene, substituted alkynylene, heteroalkylene, substituted heteroalkylene, heteroarylene, substituted heteroarylene, cycloalkylene, substituted cycloalkylene, heterocycloalkylene, substituted heterocycloalkylene, cycloalkenyl, —C(O)—, and —COO—.

16. A method for multiplexed antigen imaging and profiling in a sample, the method comprising contacting one or more DNA-barcoded antibodies of claim 14 with the sample, and imaging the multiplexed antigens in the sample.

17. The method for multiplexed antigen imaging and profiling of claim 16, wherein imaging the multiplexed antigens in the sample uses fluorescent microscope techniques selected from confocal microscopy, two- or multi-photon microscopy, light sheet microscopy, and super-resolution microscopy techniques.

18. The method for multiplexed antigen imaging and profiling of claim 16, wherein the step of contacting takes 10 minutes at room temperature.

19. A method for multiplexed biomolecule detection in a sample, the method comprising contacting one or more DNA-barcoded antibodies of claim 14 with the sample, and imaging the multiplexed biomolecules in the sample.

20. A method for identifying cells that express a biomarker of interest in a sample, the method comprising contacting the DNA-barcoded antibody of claim 14 with the sample, and identifying cells that express the biomarker of interest.
